(19) United States
(12) Patent Application Publication
(10) Pub. No.: US 2002/0182239 A1
(43) Pub. Date: Dec. 5, 2002

(54) Composition and method for inhibiting polar capsule discharge and protecting a subject from nematocyst sting

(76) Inventor: Amit Lotan, Jordon Valley (IL)

Correspondence Address: Eitan Pearl Latzer & Cohen-Zedek, One Crystal Park Suite 210, 2011 Crystal Drive, Arlington, VA, 22202-3709, US

(21) Appl. No.: 10/148,258

(22) PCT Filed: Nov. 29, 2000
(86) PCT No.: PCT/IL00/00805

(51) Int. Cl. A61K 33/24; A61K 33/14; A61K 7/00

(52) U.S. Cl. 424/401; 424/617; 424/678; 424/681

(57) ABSTRACT

This invention provides a composition comprising formalin and chloride. In another aspect of the invention, the composition may also comprise magnesium chloride, calcium chloride, or lanthanum chloride. This invention provides a composition comprising formalin and lanthanum chloride. This invention provides an ointment which comprises a composition comprising formalin and chloride. This invention provides a method of inhibiting nematocyst or polar capsule discharge and a method of protecting a subject from a sting from an organism discharging nematocyst or polar capsule discharge comprising applying an effective amount of the composition of comprising formalin and chloride, to the skin of a subject prior to contact with nematocysts or polar capsules, thereby inhibiting nematocyst or polar capsule discharge.

### FIELD OF THE INVENTION

The present invention relates to a composition and method for inhibiting nematocyst or polar capsule discharge and protecting a subject from nematocyst sting. The composition can be in the form of an emulsion, liquid dispersion, spray, solution, skin cream, face cream, lotion or ointment.

### BACKGROUND OF THE INVENTION

Swimmers and divers throughout the world are familiar with the stinging organisms of the oceans such as jellyfish, sea anemone and coral. Jellyfish stings, although seldom fatal, are a major public health problem. Lotan et al. (1992) Marine Biology 112:237-242; Lotan et al. (1994) Marine Ecology Progress Series 109:59-65. In the summer months, it is estimated that over 500,000 swimmers and divers in the Chesapeake Bay area and 200,00 persons in Florida are stung by jellyfish, mainly by “sea nettles,” the common name of Chrysaora quinquecirrha. Burnett et al. (1992) MMI 41(6):509-513. Similarly, between the months of March and August, one in four Florida bathers are stung and more than 10,000 persons require emergency medical treatment for pruritic eruptions caused by contact with jellyfish larvae known as “sea lice.” Tomchik el al. (1993) JAMA269(13):1669-1672.

The members of the phylum Cnidaria (e.g., jellyfish, sea anemone and coral) and the phylum Myxozoa are all equipped with stinging subcellular organelles, known as nematocysts, cnidocysts, or polar capsules. The nematocysts are located in specialized cells (nematocytes) and consist of a capsule containing a condensed tubule with potent toxins and threads. When nematocysts discharge, the tubule penetrates into its target organism and releases its toxins. The threads arrayed on the tubule enhance the anchoring and attachment of the nematocyst tubule to its target. Thus, nematocysts are involved in target recognition, toxin delivery, infection and attachment.

All members of the phyla Cnidaria and Myxozoa contain nematocysts of varying sizes, shapes and types Mariscal, pp 129-178, “Nematocysts” in COELENTERATE BIOLOGY: REVIEWS AND NEW PERSPECTIVES, eds. Muscatine and Lenhoff (Academic Press, New York, 1974). These different types of cnidocysts function in diverse biological roles including capture of prey, toxin delivery, recognition, attachment, adherence and infection. (see, e.g., Tardent (1995); Bioessays 17(4):351-362; Lotan et al. (1995) Nature 375:4A56; Lotan et al. (1996) Expt'l Zool. 275:444-451; Lotan (1996) et al., pp. 132-145, “Toxicology and ecology and the Mediterranean jellyfish Rhopileim nomadica” in BIOCHEMICAL ASPECTS OF MARINE PHARMACOLOGY, eds. Lazarovici et al. (Alaken, Inc., Fort Collins, Colorado, 199Q; Spaulding (1972) Biol. Bull. 143:440453; Holstein and Tardent (1984) Science 223:830-833 and Mariscal, supra.

Conventionally, nematocysts stings have been treated with antidotes such as steroids, aluminum sulfate/surfactant and antihistamines. Tomchik, supra. For example, Wasuwat (1970) Nature 225:758, descibes how Thai fisherman use an extract made from the leaves of pomoea pes-caprae as an antidote to jellyfish poison. When the extract was analysed, it was found to be mildly antihistaminic. The extract exhibits the same effect against jellyfish poison as two commercially prepared antidotes containing antihistamines. In Australia, vinegar is recommended for treatment of lethal box jellyfish stings. Hartwick et al. (1980) Med. J Aust 1: 15-20. Vinegar is not recommended for stings by other species. Fenner et aL (1993) Med. J Aust. 158:498-501.

Since post-sting treatments of nematocyst stings are often unsatisfactory, the search for ways to prevent nematocyst discharge has been ongoing. The most often prescribed method of preventing jellyfish stings is avoiding any contact with the nematocysts. Tomchik, supra. However, in the case of microscopic larvae, this often means foregoing all ocean activities during the months of high incidence, (e.g., March through August in Florida). It would, therefore, be useful to have a means for inhibiting nematocyst discharge even when contact does occur. Australian patent application 67563/94 (WO 94/17779) discloses topical hydrodispersion preparations that are reported to give protection from Actiive in preventing nematocyst discharge as measured by scanning electron microscopy (SEM). The formulations contain inorganic micropigments incorporated into the lipid phase of the hydrodispersion, an optional UV filtering substance and are essentially free of emulsifiers. Lubbock (1979) J Eyp. BY. 83:283-292 describes how proteinaceous compounds tend to induce a stronger response leading to nematocyst discharge in sea anemones than either polysaccharides or lipids. The authors could determine no simple recognition basis and speculated that the process of nematocyst discharge was complex. Lubbock and Amos, supra, disclose that isolated nematocyst capsules do not discharge in 50 mM CaCl2. The authors report that inhibition of isolated nematocyst discharge occurs only if a solute that could not rapidly penetrate the capsule wall is used, for example, high molecular weight polyethylene glycol. Thus, calcium in the surrounding environment may stabilize nematocysts because it reduces the differential between the calcium concentration outside the capsule and inside the capsule. Normally, the calcium concentration inside the nematocyst capsule is approximately 600 mM. Normal calcium concentration in sea water is around 7 mM, about 100 fold less than inside the capsule. Thus, increasing the calcium concentration outside the capsule to 50 mM reduces the differential to around 10 fold and may be involved in inhibiting nematocyst discharge.

Heeger et al. (1992) Marine Biology 113:669-678 tested the ability of three commercially available sunscreen lotions to inhibit jellyfish nematocyst discharge on samples of live human skin. Two of the three lotions were effective at reducing the number of nernatocysts discharged. The authors concluded that glycerol and oil components of the lotions could be masking or suppressing the effects of natural stimuli of the skin, however, even the lotion which did not inhibit nematocyst discharge contained these substances Hartwick et al. (1980) Med J Australia 1:15-20 report that commercial sting remedies do not inhibit nematocyst discharge. Accordingly, there is a commercial need for a composition which inhibits nematocyst discharge and protects a person from being stung.

Lastly, water soluble aluminum salts are known to serve as a method for treatment of bites, stings and wounds (Australian patent 475036, 513 983). The mechanisms of the action of aluminum salt in relation to relief of bites or stings is not clear and it was suggested that the aluminum ion denatures venom or toxins and other stinging substances which may be the pain-causing agents. A composition for treatment of venomous bites and stings was developed by Douglas Henderson (Patent no. 2110534 ). This waterproof composition contains concentrations of 13%-56% of aluminum salts and Methyl Eugenol. This composition was found to be effective when applied topically against stinging from Bluebottle (Portuguese Man-o-War). The composition gave limited protection against Box jellyfish (Chronex fleckeri) stings.

### SUMMARY OF THE INVENTION

This invention provides a composition comprising formalin and chloride. In another aspect of the invention, the composition may also comprise magnesium chloride, calcium chloride, or lanthanum chloride. This invention provides a composition comprising formalin and lanthanum chloride. This invention provides an ointment which comprises a composition comprising formalin and chloride.

This invention provides a method of inhibiting nematocyst or polar capsule discharge comprising applying an effective amount of the composition comprising formalin and chloride to the skin of a subject prior to contact with nematocysts or polar capsules, thereby inhibiting nematocyst or polar capsule discharge.

This invention provides a method of protecting a subject from a sting from an organism discharging nematocyst or polar capsule discharge comprising applying an effective amount of the composition comprising formalin and chloride to the skin of a subject prior to contact with the organism, thereby protecting a subject from the sting from the organism discharging nematocyst or polar capsule discharge.

### BRIEF DESCRIPTION OF THE FIGURES

FIG. 1: Immunohistochemical stain of discharge of nematocyst within human skin. Human skin sections were exposed to jellyfish tentacles of Rhopilema nomadica. The Jellyfish stinging capsules that penetrated the skin were labeld with specific immunofluorescent antibody goat anti-rabbit FITC. Each green dot represents a stinging capsule that penetrated the skin.

FIG. 2: Demonstration of unprotected vs. jellyfish-repellent protected skin of human subject after being exposed to 20 seconds of direct contact with jellyfish tentacles.

FIG. 3: Development of skin infection after encounter with jellyfish

FIG. 4: Pain sensation after jellyfish exposure

### DETAILED DESCRIPTION OF THE INVENTION

During the stinging event the intensity of the sting symptoms is in proportion to the number of nematocysts that have penetrated the skin. This invention demonstrates that cnidaria nematocyst discharge onto human skin can be inhibited with a protective ointment base containing low concentrations of formalin.

This invention provides a composition comprising formalin and chloride. In one embodiment the composition comprises magnesium chloride, calcium chloride, or lanthanum chloride. In a preferred embodiment the composition comprises lanthanum chloride.

In one embodiment, the composition contains the following: laurylmethicone Copolyl (5%), Octyl Methoxycinnamate (7.5%), Polyglyceryl 4 Isosterat (1%), Cyclomethicone (7.5%), Cetyl dimethicone (3%), Hydrogenated castor oil (0.5%), Ceresine WAXSP 252 (1%), Mineral Oil (2%), Deionized water (67%), sodium chloride (0.5%), germaben 11(1%), lanthanum chloride (2.0%) and formaldehyde (0.1%) [Formula 1]

In another embodiment, the composition contains the following: laurylmethicone copolyl (5%), octyl methoxycinnamate (7.5%), Polyglyceryl 4 Isosterat (1%), Cyclomethicone (7.5%), Cetyl dimethicone (3%), Hydrogenated castor oil (0.5%), Ceresine WAXSP 252 (1%), Mineral Oil (2%), Deionized water (70%), sodium chloride (0.5%), germaben 11(1%), calcium chloride (1%) and formaldehyde (0.1%) [Formula 2]

The term “nematocyst” or “cnidocyst” or “polar capsule” is used in the conventional sense to refer to the subcellular stinging structures found in the nematocytes or cnidocytes. Nematocysts are described in detail in Tardent, supra and Mariscal, supra. These stinging organelles are capable of penetrating, anchoring onto and injecting a substance such as a toxin into a target organism.

The terms “nematocyte” or “cnidocyte” or “cnidoblast” are intended to refer to the specialized cell which contain the nematoeysts. Non-limiting examples of classes of the phylum Cnidaria which possess cnidocytes include, for example, Hydrozoa, Anthozoa, Myxozoa and Scyphozoa. Examples of jellyfish include, but are not limited to, Aurelia sp., Pelagia sp., Chrysaora sp., Anthoplaura sp., Rhopilema sp., Physalia sp., Cyanea sp., Linuche sp., Catostylus sp., Carybdea sp., Chironex sp., Stomolophus sp., Rhiozostoma sp., Corinactis sp. and the like.

The term “final concentration” refers to the concentration of the ingredient after the composition has been prepared, including, for instance, the concentration of the composition after it has been placed in an aqueous environment.

The composition comprising formalin and chloride or formalin and lanthanum chloride or formalin, lanthanum chloride and/or magnesium chloride, calcium chloride may is incorporated into various formulations. Such formulations include but are not limited to: emulsions, sprays, liquid dispersions, solutions, skin creams, face creams, lotions or ointments. Such compositions may contain emulsifiers, water, emollients, dry-feel agents, waterproofing agents, preservatives, antioxidants, anti-foaming agents and fragrances as well as any other class of materials whose presence may be desirable, which are known to those skilled in the art.

This invention provides a method of inhibiting nematocyst or polar capsule discharge comprising applying an effective amount of the composition containing one or more of the following: a) formalin and chloride, or b) formalin and lanthanum chloride, or c) formalin, lanthanum chloride and/or magnesium chloride to the skin of a subject prior to contact with nematocysts or polar capsules, thereby inhibiting nematocyst or polar capsule discharge.

This invention provides a use of a compositions containing one or more of the following: a) formalin and chloride, or b) formalin and lanthanum chloride, or c) formalin, lanthanum chloride and/or magnesium chloride for inhibiting nematocyst or polar capsule discharge comprising applying an effective amount of the composition to the skin of a subject prior to contact with nematocysts or polar capsules, thereby inhibiting nematocyst or polar capsule discharge.

This invention provides a method of protecting a subject from a sting from an organism discharging nematocyst or polar capsule discharge comprising applying an effective amount of the composition comprising one or more of the following: a) formalin and chloride, or b) formalin and lanthanum chloride, or c) formalin, lanthanum chloride and/or magnesium chloride to the skin of a subject prior to contact with the organism, thereby protecting a subject from the sting from the organism discharging nematocyst or polar capsule discharge.

This invention provides use of a composition comprising an effective amount of one or more of the following: a) formalin and chloride, or b) formalin and lanthanum chloride, or c) formalin, lanthanum chloride and/or magnesium chloride for protecting a subject from a sting from an organism discharging nematocyst or polar capsule discharge by applying, to the skin of a subject prior to contact with the organism, thereby protecting a subject from the sting from the organism discharging nematocyst or polar capsule discharge.

As provided herein, nematocysts are discharged from an organism selected from the group consisting of Aurelia sp., Pelagia sp., Chrysaora sp., Anthoplaura sp, Rhopilema sp., Physalia sp., Cyanea sp., Linuche sp., Catostylus, Carybdea sp., Chironex sp., Stomolophus sp., Rhiozostoma, Corinactis sp., Aurelia aurita, Corynactic californica, Anthopleura elegantissima, Pelagia noctiluca., Chrysaora quinquecirrha and Anthoplaura sp., Hydrozoa, Anthozoa or Schyphoza and Myxozoan sp.

Emulsions/Emulsifiers

An emulsion is a mixture of two immiscible liquids, i.e. liquids that are not mutually soluble, but are mechanically agitated and shaken so thoroughly together that one liquid forms drops in the other one, giving the mixture the appearance of a homogeneous liquid. If the emulsifier is added to the two immiscible liquids, one of them becomes continuous and the other one remains in droplet form. As used herein in reference to the water-in-oil emulsifiers, the term “HLB value” means the hydrophilic-lipophilic balance. The HLB value has been used by those skilled in the emulsion art for selecting emulsifiers useful for preparing water-in-oil emulsions. See U.S. Pat. No. 4,177,259 and references cited therein.

An oil-in-water (o/w) emulsion is a mixture in which oil droplets (the discontinuous phase) are dispersed in water (a continuous aqueous phase). A water-in-oil (w/o) emulsion is a mixture in which water droplets (the discontinuous phase) are dispersed in oil (a continuous oil phase). The type of emulsion, oil-in-water (o/w) or water-in-oil (w/o), is determined by the volume ratio of the two liquids, provided the ratio is sufficiently high. For example, with 5% water and 95% oil (an o/w phase ratio of 19), the emulsion likely will become w/o. For moderate ratios (<3), the type of emulsion is decided by several factors, such as order of addition or type of emulsifier. One liquid slowly added to a second liquid with agitation usually results in the second liquid being the continuous phase. Another factor is preferred solubility of the emulsifier, the phase in which the emulsifier is soluble most probably is continuous.

More complex emulsions, such as double emulsions, are formed when an emulsion is dispersed in an continuous phase. For example, in an oil in-water-in oil (o/w/o) emulsion, the water in a continuous water phase containing dispersed oil droplets, which are themselves dispersed in a continuous oil phase. Similarly, in a water-in oil-in water emulsion, the oil in a continuous phase containing dispersed water droplets, which are themselves dispersed in a continuous water phase. These more complex emulsions find use as a system for slow delivery, extraction, etc.

An emulsifier (a stabilizing compound) is an agent used to assist in the production of an emulsion. Typically, emulsifiers are molecules with non-polar and polar parts that are able to reside at the interface of the two immiscible liquids.

The compositions described herein may also be combined with sunscreens. Such sunscreens are also known to those skilled in the art. Typical sunscreen active ingredents include trade names of para-aminobenzoic acid up to about 15 weight percent or from about 5 to 15% in admixture with other sunscreen actives; cinoxate up to about 3 weight percent or about 1 to 3% in admixture; diethanolamine methoxycinnamate up to 10 weight percent or about 8 to 10% in admixture; digalloyl trioleate up to 5 weight percent or about 2 to 5% in admixture; dioxybenzone up to 3 weight percent alone or in admixture; ethyl 4[bis(hydroxypropyl)]aminobenzoate up to 5 weight percent or about 1 to 5% in admixture; glyceryl aminobenzoate up to 3 weight percent or about 2 to 3% in admixture; homosalate up to 15 weight percent or about 4 to 15% in admixture; lawsone up to 0.25 weight percent with dihydroxyacetone up to 3 weight percent; menthyl anthranilate up to 5 weight percent or about 3 to 5% in admixture; octocrylene up to 10 weight percent or 7 to about 10% in admixture; octyl methoxycinnamate up to 7.5 weight percent or about 2 to 7.5% in admixture; octyl salicylate up to 5 weight percent or about 3 to 5% in admixture; oxybenzone up to 6 weight percent or about 2 to 6% in admixture; padimate up to 8 weight percent or about 1.4 to 8% in admixture; phenylbenzimidazole sulfonic acid up to 4 weight percent or about 1 to about 4% in admixture; red veterinary petrolatum up to 95 percent or about 30 to 95% in admixture; sulisobenzone up to 10 weight percent or about 5 to 10% in admixture; titanium dioxide up to 25 weight percent or about 2 to 25% in admixture; and trolamine salicylate up to 12 weight percent or about 5 to 12% in admixture. Typical suitable UV-B type sunscreening active ingredients include octyl para-methoxycinnamate available from Givaudan Corporation, Clifton, N.J., under the trade name of Parsol MCX and Parsol 1789, usually present in the range of about 1.5 to about 7.5 weight %, and octyl salicylate available from Harmann and Riemer, Springfield, N.J., 07081, usually in the range of about 3 to about 5 weight %, of the total sunscreen composition or emulsion. The amount of UV-B type sunscreening active ingredients should be sufficient to give an SPF of at least 2 to 15. A typical suitable UV-A type sunscreening actives include benzophenone-3, usually in the range of about 0.5 to about 6 weight %. Such active can be obtained from Rhone-Poulenc, Atlanta, Ga. Sunscreen emulsions containing mixtures of UV-B and UV-A type sunscreen actives should be sufficient to provide an SPF of 4 to 50. water Water may be used as a diluent or can be the internal (discontinuous) or external (discontinuous) phase of an emulsion system. An emollient is an oil-containing agent which helps to smooth and soften the skin, and may also reduce its roughness, cracking or irritation. Typical suitable cosmetic emollients include mineral, oil, having a viscosity in the range of 50 to 500 SUS, lanolin oil, coconut oil, cocoa butter, olive oil, almond oil, macadamia nut oil, aloe extract, jojoba oil, safflower oil, corn oil, liquid lanolin, cottonseed oil and peanut oil. Other typical suitable cosmetic emollients include Purcellin oil, perhydrosqualene, castor oil, polybutene, odorless mineral spirits, sweet almond oil, avocado oil, calophyllum oil, ricin oil, vitamin E acetate, olive oil, silicone oils such as dimethylopolysiloxane and cyclomethicone, linolenic alcohol, oleyl alcohol, the oil of cereal germs such as the oil of wheat germ, isopropyl palmitate, isopropyl myristate, hexadecyl stearate, butyl stearate, decyl oleate, acetyl glycerides, the octanoates and benzoates of (C.sub.12-C.sub.15) alcohols, the octanoates and decanoates of alcohols and polyalcohols such as those of glycol and glycerol, ricin oleates of alcohols and poly alcohols such as those of isopropyl adipate, hexyl laurate and octyl dodecanoate. Other typical suitable cosmetic emollients which are solids or semi-solids at ambient temperatures may be used if admixed with mineral oil or extra heavy mineral oil in amounts sufficient to provide liquid topical compositions. Such solid or semi-solid cosmetic emollients include hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. Typical suitable emollients include esters of a straight or branched-chain C.sub.10-C.sub.16 alcohol and a straight or branched chain C4-C20 mono- and dicarboxylic acids include the straight and branched chain monocarboxylic acids substituted by hydroxy or double bonds including monocarboxylic acids such as: butanoic, pentanoic, 2-methyl- and 3-methyl-pentanoic, 2,2-dimethylpropanoic, hexanoic, 2-methyl-, 3-methyl-, and 4-methyl-, 5-methyl- and 6-methyl-heptanoic, 2-ethylhexanoic, octanoic (caprylic), 2-methyl-, 3-methyl-, 4-methyl-, 5-methyl-, 6-methyl-, 7-methyl- and 8-methyl-nonanoic, 3,3,5-trimethylexanoic (isonanomic), decanoic (caproic), 2-methyl-, 3-methyl-, 4-methyl-, 5-methyl-, 6-methyl-, 7-methyl-, 8-methyl-, 9-methyldecanoic, undecanoic, dodecanoic (lauric), dineopentylacetic, methyl-tbutyineopentylacetic, tridecanoic, tetradecanoic (myristic), pentadecanoic, hexadecanoic (palmitic), heptadecanoic (margaric), octadecanoic (stearic), hydroxystearic 16-methylheptadecanoic (isistearic), double bond subsituted (unsaturated) carboxylic acids such as oleic(cis-9-octadecenoic), linoleic(cis, cis-9,12-octadecadienoic) and linolenic cis,cis,cis-9,12,15-octadecadienoic acid), nonadecanioc and CH.sub-3 (CH.sub.2).sub.18 CO.sub.2 H.; Typical suitable C.sub.4-C.sub.20 dicarboxylic acids include dicarboxylic acids of the formula (CH.sub.2).sub.n (CO.sub.2 H).sub.2 wherein n is 2 to 18 including succinic (n=2), glutaric (n=3), adipic (n=4), pimelic (n=5), suberic (n=6), azelaic (n=7), sebacic (n=8) as well as the C.sub.12, C.sub.16 and C.sub.19 members such as Brassilic (C. sub.13), thapsic (C.sub.16) and nonadecance-1,19-dicarboxylic acid. The preferred dicarboxylic acids are succinic and adipic.

Typically suitable tri (lower alkyl) substituted benzoic acids include trimethylbenzoic acids, such as 1,1,3-trimthylbenzoic acid, 1,2, 4-trimethyl-benzoic acid (trimellitic acid), and 1,3,5-trimethylbenzoic acid (trimesic acid). The preferred tri (lower alkyl) benzoic acid is trimellitic acid. Preferred esters of C.sub.4 to C.sub.20 monocarboxylic acids and straight and branched chain C10-C16 alcohols include tridecyl neopentonate, isotridecyl isononanoate, isodecyl neopentonate, isodecyl hydroxystearate, isodecyl laurate, isodecyl myristate, isodecyl oleate, isodecyl palmitate, decyl oleate, and isocetyl palmitate (isohexadecyl hexadecanoate) and iso-hexadecyl isodecanoate (14-methylpentadecyl-8-methylnonanoate). The preferred ester of straight and branched-chain C10-C16 alcohols and C4-C20 dicarboxylic acids is decyl succinate. The preferred esters of straight and branched-chain C10-C16 esters of tri(loweralkyl) substituted benzoic acids are the decyl, isodecyl, isotridecyl and tridecyl esters of trimethylbenzoic acids especially trimellitic acid; tridecyl trimellitate is more preferred.

The emollient can be a mixture of tridecyl trimellitate and tridecyl stearate or a mixture of tridecyl trimellitate, tridecyl stearate, neopentyl glycol dicaprylate and neopentyl dicaprate such as is available from Lipo Chemicals Inc., Patterson, N.J. under the trade name Lipovol MOS-70. The emollient component can optionally be included in the sunscreen composition in an amount ranging from about 10 to about 50 weight percent, preferably about 20 to about 40 percent. A dry-feel modifier is an agent that gives a sunscreen a “dry feel” upon drying, and may reduce sunscreen migration and can include silazane treated silica, precipitated silica, fumed silica or mixtures thereof. The dry-feel modifier can optionally be included in the sunscreen composition in amounts ranging from about 0.1 to about five weight percent, preferably from about 0.3 to about 1.5%.

A waterproofing agent is a material added to the composition to assist in retaining the sunscreen active ingredients on the skin. Typical suitable waterproofing agents for sunscreen raw materials include copolymers derived from polymerization of octadecene-1 and maleic anhydride in accordance with the published procedures such as those in U.S. Pat. No. 3,860,700 and Reissue No. 28,475. The preferred waterproofing agent is a copolymer commercially available from Chevron Chemicals Co. under the trade name, PA-18 polyanhydride resin. The term “waterproofing effective amount of at least one waterproofing agent” means that the waterproofing agent(s) is applied in amounts effective to allow the sunscreen to remain on the skin after exposure to circulating water for at least 80 minutes using the procedures described in “Sunscreen Drug Products for OTC Human Use”, Federal Register, Vol. 43, Aug. 25, 1978, Part 2, pages 38206-38269. The waterproofing agent can optionally be included in the sunscreen composition in an amount ranging from about 0.01 to about 10.0 weight percent, preferably about 1.0 to about 10.0 percent.

An antimicrobial preservative is a substance or preparation that destroys, prevents or inhibits the multiplication/growth of microorganisms in the sunscreen composition and may offer protection from oxidation. Preservatives are used to make aqueous products self-sterilizing. This is done to prevent microorganisms that may be in the product from growing during manufacturing and distribution of the product and during use by consumers who may inadvertently contaminate the product during normal use. Typical preservatives include the lower alkyl esters of para-hydroxybenzoates (parabens) especially, methyl paraben, isobutyl paraben and mixtures thereof, and benzoic acid. The antimicrobial preservative can optionally be included in the sunscreen composition in an amount ranging from about 0.05 to about one percent, preferably about 0.2 to about 0.5 percent. One exemplary preservative is Germaben II, trade name of Sutton Labs, Chatham, N.J.

An antioxidant is a natural or synthetic substance added to the sunscreen to prevent or delay its deterioration due to the action of oxygen in the air (oxidation). Anti-oxidants prevent oxidative deterioration which may lead to the generation of rancidity and nonenyzymatic browning reaction products. Typical suitable antioxidants include propyl, octyl and dodecyl esters of gallic acid, butylated hydroxyanisole (BHA)(usually as a mixture of orthos and meta isomers), butylated hydroxytoluene (BHT) and nordihydroguaiaretic acid. The antioxidant can optionally be included in the sunscreen composition in an amount ranging from about 0.02 to about 1.0 weight percent, preferably about 0.05 to about 0.1 percent.

Fragrances are aromatic compounds that can impart an aesthetically pleasing aroma to the sunscreen composition. Typical fragrances include aromatic materials extracted from botanical sources (i.e. rose petals, gardenia blossoms, jasmine flowers, etc.), which can be used alone or in any combination to create essential oils. Alternatively, alcoholic extracts may be prepared for compounding fragrances. The fragrance can optionally be included in the sunscreen composition in an amount ranging from about 0.01 to about 5.0 weight percent, preferably about 0.1 to about 2.0 percent. Definitions and suppliers of the ingredients used in the following illustrative examples may be found in the CTFA Cosmetic Ingredient Dictionary, published by the Cosmetic, Toiletry and Fragrance Association, Inc., 1110 Vermont Avenue, NW, Wash. D.C. 20005, Third Edition 1982. All proportions are by percent weight.

Further, this invention provides additional agents and/or compounds which are antibacterial metal salts, salts of aluminum, zirconium, zinc, silver, gold, copper, tin, mercury, bismuth, selenium, strontium, scandium, yttrium, cerium, praseodymium, neodymium, promethium, samarium, europium, gadolinium, terbium, dysprosium, holmium, erbium, thulium, ytterbium, lutetium and mixtures thereof. Preferred antimicrobial agents for use herein are the broad spectrum actives selected from the group consisting of Triclosan, Triclocarban, Octopirox, PCMX, ZPT, natural essential oils and their key ingredients, and mixtures thereof. The most preferred antimicrobial active for use in the present invention is Triclosane.

Lipophilic skin-moisturizing agent: Lipophilic skin-moisturizing agents are preferably included in the compositions herein in an amount ranging from about 0.1% to about 30%, preferably from about 1% to about 30% more preferably from about 3% to about 25%, most preferably from about 5% to about 25% of the composition.

A wide variety of lipid type materials and mixtures of materials are suitable for use as the carrier in the antimicrobial personal cleansing compositions of the present invention.

Preferably, the lipophilic skin-conditioning agent is selected from the group consisting of hydrocarbon oils and waxes, silicones, fatty acid derivatives, cholesterol, cholesterol derivatives, di- and tri-glycerides, vegetable oils, vegetable oil derivatives, liquid nondigestible oils such as those described in U.S. Pat. No. 3,600,186 to Mattson, Issued Aug. 17, 1971, and U.S. Pat. No. 4,005,195 and 4,005,196 to Jandacek et al., both issued Jan. 25, 1977, all of which are herein incorporated by reference, or blends of liquid digestible or nondigestible oils with solid polyol polyesters such as those described in U.S. Pat. No. 4,797,300 to Jandacek; issued Jan. 10, 1989, and U.S. Pat. Nos. 5,306,514, 5,306,516 and 5,306,515 to Lefton, all three issued Apr. 26, 1994, all of which are herein incorporated by reference, and acetoglyceride esters, alkyl esters, alkenyl esters, lanolin and its derivatives, milk triglycerides, wax esters, beeswax derivatives, sterols, phospholipids and mixtures thereof. Fatty acids, fatty acid soaps and water soluble polyols are specifically excluded from our definition of a lipophilic skin-moisturizing agent.

Hydrocarbon oils and waxes: Some examples are petrolatum, mineral oil microcrystalline waxes, polyalkenes (e.g. hydrogenated and nonhydrogenated polybutene and polydecene), paraffins, cerasin, ozokerite, polyethylene and perhydrosqualene. Blends of petrolatum and hydrogenated and nonhydrogenated high molecular weight polybutenes wherein the ratio of petrolatum to polybutene ranges from about 90:10 to about 40:60 are also suitable for use as the lipid skin-moisturizing agent in the compositions herein.

Topical Formulations

The compositions and/or formulations may be topically applied to a subject. Preferably, topical formulations containing compositions within the present invention create physical barriers to minimize skin stimulant secretion and are, themselves, riot stimulants. Formulations should be water-proof so as to be useful for swimmers and divers. Ca+ binding compounds such as sodium citrate, EDTA, amino acids and glycoproteins generate nematocysts discharge and are, therefore, not to be used. Examples of suitable carriers include, but are not limited to, dextran, dextran sulfate, agarose, phosphatidyl ethanolamine, cholesterol, cholesterol palmitate, palmitic acid, oleic acid, lysolecitin, phosphatidyl choline, phosphatidyl serine, phosphatidyl inositol, squalene, phenyl trimethicone, stearic acid, propylene glycol sterate, petrolatum, stearoxytrimethyl-silane, stearyl alcohol. Other carriers are known to those skilled in the art. Silicone polymers are preferred as they provide good barriers, are water-proof and do not stimulate nematocyst discharge. Optionally, the compositions described herein may contain a known sunscreen. Preferably, the sunscreens contain at least one UVA filter and at least one UVB filter. Oil-soluble UVB products include 3-benzylidenecamphor derivatives, 4-aminobenzoic acid derivatives, esters of cinnamic acid, derivatives of benzophenone, esters of benzylidenemalonic acid Water soluble UVA filters include salts of 2-phenylbenzimidazole-5-sulphonic acid, sulphonic acid derivatives of benzophenones and sulphonic acid derivatives of 3-benzylidenecamphor. Effective amounts of sunscreens will be known to those of skill in the art. Preferably, sunscreens will comprise between about 0.1% and 10% by weight of the total preparation.

The following examples are intended to illustrate but not to limit the subject invention.

### Experimental Details Section

### EXAMPLE 1

### Large Number of Stinging Cells are Discharged During the Stinging Event

To test the ability of formalin to inhibit nematocyst discharge, a test using human skin was performed as described herein. Normal human skin was obtained from neonatal elective circumcision. The skin was immediately placed into sterile phosphate buffered saline (PBS) containing antibiotic/antimycotic. The tissue was frozen and embedded in Tissue-Tek™. It was then sectioned into 50 μm sections using a cryostat. The sections were attached to superfrost/plus microscope slide (Fisherbrand) and maintained at −20° C. until time of use. All sections were examined to ensure that they were perpendicular to the plane of the epidermis, exposing cross-sections of epidermis, dermis and sub-coetaneous tissue. Immunoassay tests to detect cnidaria stinging level into human skin: Polyclonal antibody specific for nematocyst toxin was prepared, as described in Lotan et al (1996).

The HPLC fraction containing the toxin phospholipase A2 from the jellyfish Rhopilema nomadica was isolated and injected into rabbits as described in Vaitukaitis, pp. 46-52, “Immunochemical Techniques, Part B,” in Methods in enzymology, vol. 73, ed. Larson & Vunkis (academic Press, New York, 1981). Serum was collected from the rabbits after two injections, and the presence of antibody against the jellyfish toxin was examined on immunoblots.

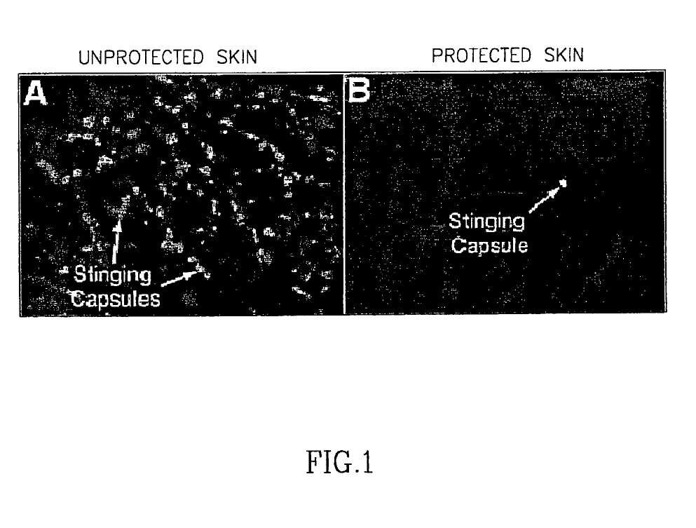

The slides prepared and frozen as described above were thawed at room temperature. The slides were submersed in sea water and then attached to jellyfish or sea anemone tentacles for 10 seconds. The tentacles were detached, the slide washed with PBS, and blocked using 5% Fetal Bovine Serum, and 0.1% Triton X100 in PBS (blocking solution). All washing incubation steps were conducted on a tilting shaker at room temperature. The slides were incubated at 10 ml of block solution containing a 1:2, 500 dilution of the anti-toxin antibody for one hour. After incubation, the slide were washed five times for five minutes each wash with 100 mL of 0.1% TritonX100 in PBS (Wash solution). The slides were then incubated in 10 mL blocking solution containing a second antibody, Goat anti rabbit FITC (Fluorescent marker) for 30 minutes. After incubation, the slides were washed five times for five minutes each wash with Wash solution. The excess PBS was removed by shaking the slide. One drop of 10% glycerol was added to each slide and the slides were covered with glass coverslips. The slides were observed under a conventional light microscope or under an ultraviolet (UV) microscope at least at 200× magnification (See FIG. 1).

### EXAMPLE 2

### Preparation of Ointment Base for Testing Inhibitory Effect of Formalin as a Skin Protectant Against Cnidaria Stings.

To test the effect of formalin as a sting protectant against jellyfish sting the following ointment bases were prepared:

Twenty ml of mineral oil was mixed and heated to 65° C. with 2 gram each of Silicon cosmetic (Dow Corning 2503) and Laurylmethicone Copolyol until clear solution was observed. Under slow mixing 56 mL of water with 0.5 gram of NaCl was added. The emulsion was divided into two tubes: Tube1, 10 mL of 0.5% formalin was added to create final concentration of 0.1% formalin in the emulsion. Tube2 served as a control and only 10 ml of water was added to the emulsion. After slow mixing, emulsion was formed in the tubes.

To monitor the influence of 0.1% formalin ointment base on nematocyst discharge into human skin the jellyfish Rhopilema nomadica and the sea anemone Actinia were used in this experiment.

Twenty sections of human skin samples were tested. The sections were marked and lotion was applied randomly with ointment bases with and without 0.1% formalin. The samples were submersed in seawater and then attached to jellyfish tentacles for 10 seconds. The number of nematocysts that penetrated the skin was determined using immunohistochemical analysis.

The average number of nematocysts that were counted on a skin sample that was treated with the ointment base alone was 27+/−10 nematocysts. The average number of nematocyst that were counted on a skin sample that was treated with the ointment base containing 0.1% of formalin was 5+/−4 nematocysts. These results show that skin treated with ointment base alone contained more than 5 times more metocysts than skin that is treated with 0.1% formalin. Therefore, in the presence of formalin there is a dramatic decrease (over 80%) in nematocysts skin penetration. These results demonstrate that 0.1% formalin is significantly effective in inhibiting nematocyst discharge into human skin. Similar results were obtained with the sea anemone Actinia. Further tests to show the influence of formalin concentration on the inhibition of stings demonstrate that lower concentration of formalin (0.01%, 0.05%) reduces nematocyst discharge and skin penetration.

### EXAMPLE 3

### Protection Revel of Compositions of Formalin with Cations Against Jellyfish

Laboratory experiments were conducted on 18 sections of human skin which was treated with cream having a combination of formalin and lanthanum chloride. The skin was subjected to jellyfish tentacles for 20 seconds. An Immunofluorescent technique was used to monitor the number of nematocysts that penetrated into the skin. No nematocysts were observed on the lotioned samples. Similar results were obtained with combinations of formalin with calcium chloride and formalin with magnesium chloride.

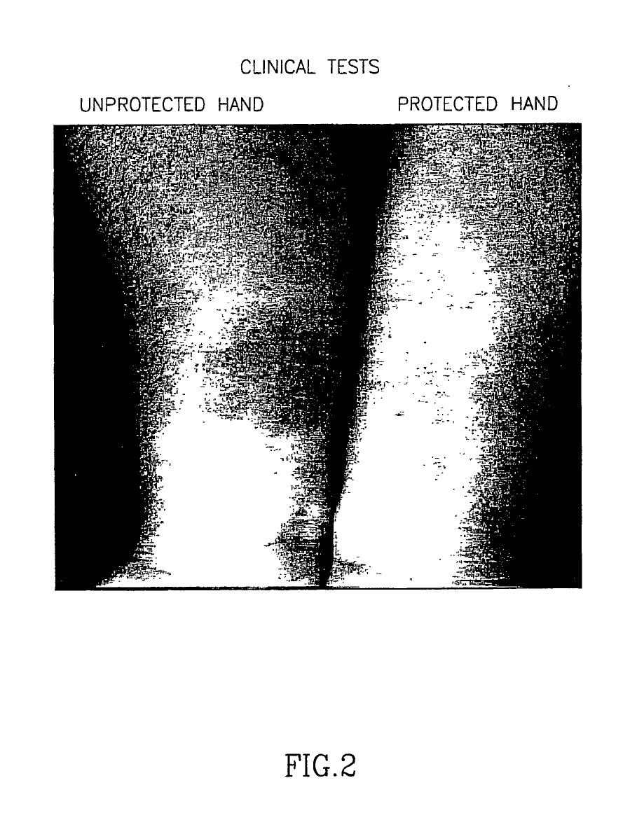

The results of the protection using a composition having formalin and lanthanum chloride on human subjects is shown in FIG. 2.

Formalin and lanthanum chloride was prepared as a cream. For example see Formula 1 as described above. This cream was applied on six inner arms of human subjects. Tentacles of the jellyfish Rhopilema nomadica were laid on the lotioned area for 20 seconds. No infection or pain was observed.

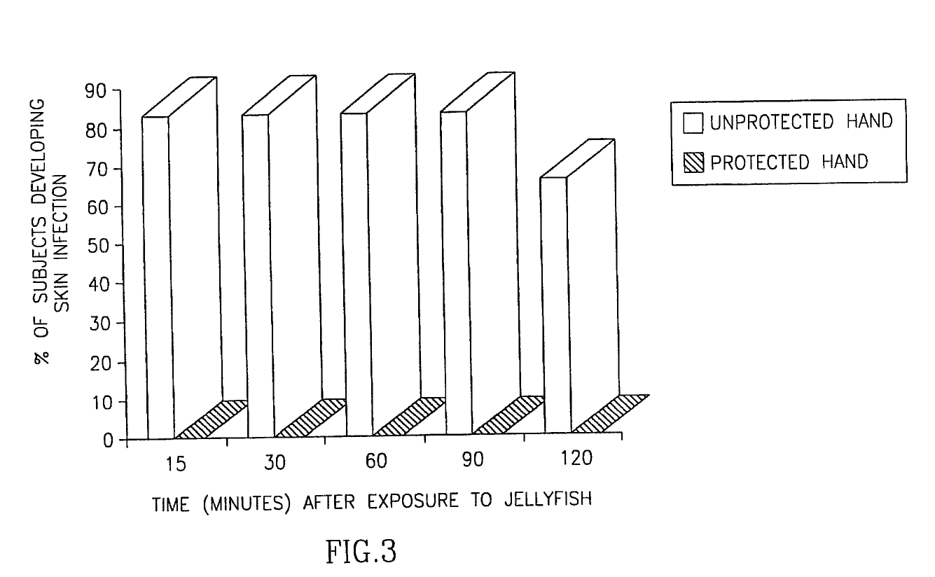

Subjects developed rash, infection and skin irritation that lasted from several hours to several days only on the control unprotected arm. (FIG. 3)

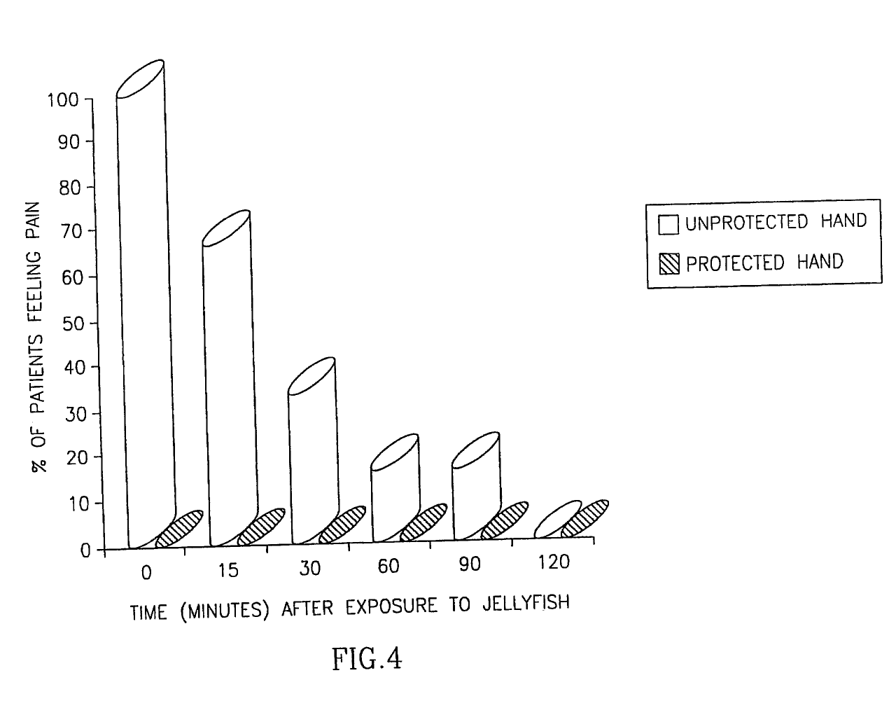

Control experiments were conducted on the same human subjects on the other inner arm. The jellyfish sting affected all the human subjects. They reported pain immediately after the tentacles were put on the unlotioned arm. After about 30 mins., rash started to develop. (FIG. 4)

What is claimed is:

1. A composition comprising formalin and chloride.

2. The composition of claim 1, wherein the chloride is magnesium chloride, calcium chloride, or lanthanum chloride.

3. The composition of claim 1, wherein the chloride is lanthanum chloride.

4. The composition of claim 3, wherein the composition further comprises magnesium chloride or calcium chloride.

5. The composition of claim 3, wherein the composition further comprises magnesium chloride and calcium chloride.

6. The composition of claim 3, wherein the composition is in the form of an emulsion.

7. The composition of claim 3, wherein the composition is in the form of a oil-in-water emulsion.

8. The composition of claim 3, wherein the composition is in the form of an emulsion, spray, liquid dispersion, solution, skin cream, face cream, lotion or ointment.

9. The composition of claim 1, wherein the composition is incorporated into an ointment base.

10. The composition according to claim 9, wherein the ointment base comprises a lipid.

11. The composition according to claim 9 wherein the ointment base comprises a silicone polymer.

12. The composition according to claim 9 further comprising a sunscreen.

13. The composition according to claim 1, wherein the final concentration of the formalin is from about 0.0005% to about 2.0%.

14. The composition according to claim 13, wherein the final concentration of the formalin is from about 0.001% to about 0.4%.

15. The composition according to claim 14, wherein the final concentration of the formalin is from about 0.0005% to about 2.0%.

16. The composition according to claim 15, wherein the final concentration of the formalin is from about 0.01%-0.4%.

17. The composition according to claim 1, wherein the final concentration of the chloride is from about 0.0005% to about 2.0%.

18. The composition according to claim 17, wherein the final concentration of the chloride is from about 0.001% to about 0.2%.

19. The composition according to claim 18, wherein the final concentration of the chloride is from about 0.0005% to about 2.0%.

20. The composition according to claim 19, wherein the final concentration of the chloride is from about 0.01%-3.0%.

21. The composition of claim 1, wherein the composition further comprises at least one cation.

22. The composition of claim 21, wherein the cation is Ca++, K+, Na+, Mn++, Co++, Mg++, W, and Fe++.

23. The composition according to claim 21, wherein the cation is K+.

24. The composition according to claim 21, wherein the cation is Mg++.

25. The composition according to claim 21, wherein the cation is Ca++.

26. A method of inhibiting nematocyst or polar capsule discharge comprising applying an effective amount of the composition of claims 1-25, to the skin of a subject prior to contact with nematocysts or polar capsules, thereby inhibiting nematocyst or polar capsule discharge.

27. A method of protecting a subject from a sting from an organism discharging nematocyst or polar capsule discharge comprising applying an effective amount of the composition of claims 1-25, to the skin of a subject prior to contact with the organism, thereby protecting a subject from the sting from the organism discharging nematocyst or polar capsule discharge.

28. The method of claim 27, wherein the nematocysts are discharged from an organism selected from the group consisting of Hydrozoa, Anthozoa or Schyphoza.

29. The method of claim 27, wherein the nematocysts or polar capsules are discharged from a Myxozoan sp.
